(19) United States
(12) Patent Application Publication
(10) Pub. No.: US 2018/0250447 A1
(43) Pub. Date: Sep. 6, 2018

(54) BAG WITH BIOCIDE AND ABSORBENT POWDER

(71) Applicants: Antonio FOLCANDO, Foggia (IT); Alessandro DI TEODORO, Pescara (IT); EVOLUZIONE S.R.L., Roma (IT); SERVIMED S.R.L., Roma (IT)

(72) Inventors: Alessandro DI TEODORO, Pescara (IT); Ettore FOLCANDO, Pescara (IT); Francesco NAPOLITANO, Roma (IT); Enrico DE LUTIO, Roma (IT)

(21) Appl. No.: 15/759,178

(22) PCT Filed: Sep. 11, 2015
(86) PCT No.: PCT/IB2015/056968

§ 371 Date: Mar. 9, 2018

(51) Int. Cl. A61L 31/16; A61F 5/441; A61F 5/44; A61M 25/00

(52) U.S. Cl. CPC A61L 31/16; A61F 5/441; A61F 5/4404; A61M 25/0017; A61F 2005/4402; A61M 2202/0401; A61M 2202/0415; A61M 2202/0496; A61M 2210/1021; A61L 2300/404

(57) ABSTRACT

The present invention relates to a bag for biological waste, such as urine, containing inside thereof a powder apt to release a biocide when the bag is filled up with the liquid, wherein the biocide is chlorine dioxide.

### FIELD OF THE INVENTION

The present invention relates to a bag for biological waste, in particular urine, containing inside thereof a powder apt to release a biocide when the bag is filled up with the liquid. Such bag is characterized in that the biocide is chlorine dioxide.

### STATE OF ART

One of the main problems linked to the use of bags for liquid organic waste for hospital use are the risks connected to the handling of such bags, in particular in the emptying step the operators are forced to provide themselves with personal protection equipment (PPE) to lower the infection risk during the procedures for emptying and disposing the bags. Currently sodium hypochlorite is used as disinfectant agent (above all for urines), but its dangerousness forces the operator to wear personal protection equipment. Furthermore, before disposal, the sodium hypochlorite must act 24 hours by reaching at least 10 cc per litre of liquid to be disinfected. Therefore, this solution has several disadvantages.

The object of the present invention is to solve this problem by providing a bag according to claim 1.

Additional features of the bag of the subject invention are defined in the corresponding depending claims.

The present invention, by overcoming the mentioned problems of the prior art, involves several and evident advantages:

the powder included in the bag according to the present invention acts once come in contact with the liquids by transforming into chlorine dioxide and then reducing the possible microbial charge; furthermore, the handling and storage thereof are much simpler, differently from chlorine.

Chlorine dioxide is a wide spectrum biocide for the microbiological lowering (viruses, bacteria, spores, fungi and algae) and it acts with a wide range of Ph values (4/10). It is a very strong oxidant and has a marked microbicide action with respect to pathogen agents, such as bacteria and viruses. Differently from chlorine, it acts on viruses by inhibiting the formation of proteins and it is more effective above all on protozoan parasites considering that it reacts directly on the cell wall of the microorganisms. It reduces the risk for health and as disinfectant it can be compared to chlorine. The traditional method for obtaining chlorine dioxide provides that this has to be produced under controlled conditions, this implies a great danger considering the high risk solved by the use of a powder, in particular of a bi-component powder.

Thanks to the present invention all biological liquids collected in the bag, apart from solidifying due to the effect of the powder absorbent, loose the possible bacterial charge thanks to the chlorine dioxide action. In this way the waste deriving therefrom will not belong to the special waste category, even if coming from dangerous departments (for example the departments of Infectiology, Oncology, etc.) or in case of infected patients.

All biological liquid waste, such as for example urines, thoracic, abdominal drainages, ascites, etc., can be collected by this type of bag so that there will be no more contamination dangers thanks to the biocide action.

By way of example reference is made to the urine collection after instillation of antiblastic chemotherapy drugs (ACD). The disposal of such urine preferably must be made “by using bags with thickening agents allowing the closed-cycle direct removal of urines . . . ” (see page 14 of ACD Guidelines Hosp. “C.O. San Giovanni dell'Addolorata”). The bag according to the present invention, apart from thickening, has the peculiar feature of inactivating the ACD thanks to the powder releasing the biocide and in the above-mentioned case the bag according to the present invention, as it is a closed-circuit one, guarantees the maximum prevention from contaminations and it will be no more necessary to perform the traditional procedures providing the use of the dangerous sodium hypochlorite involving the use of very expensive dedicated personal protection equipment (PPE) provided by the rules (see for example page 13 ACD Guidelines Hosp. “C.O. San Giovanni dell'Addolorata”).

Furthermore, in economic terms, the hospital centres adopting such solution will cut the following expenses:

1) expense for the hospital infections;

2) expense for the allocation of personal protection equipment (PPE), currently worn by the personnel for emptying the bags into not protected containers and then in the sewerage system, and no more necessary when the bags according to the present invention will be then available;

3) expense for the disposal of the liquid biological waste which is set to zero thanks to the solidifying and biocidal effect of the molecule existing inside the bag.

Other advantages, as well as the features and use modes of the present invention will result evident from the following detailed description of some preferred embodiments, shown by way of example and not for limitative purpose, by referring to the figures of the enclosed drawings, wherein:

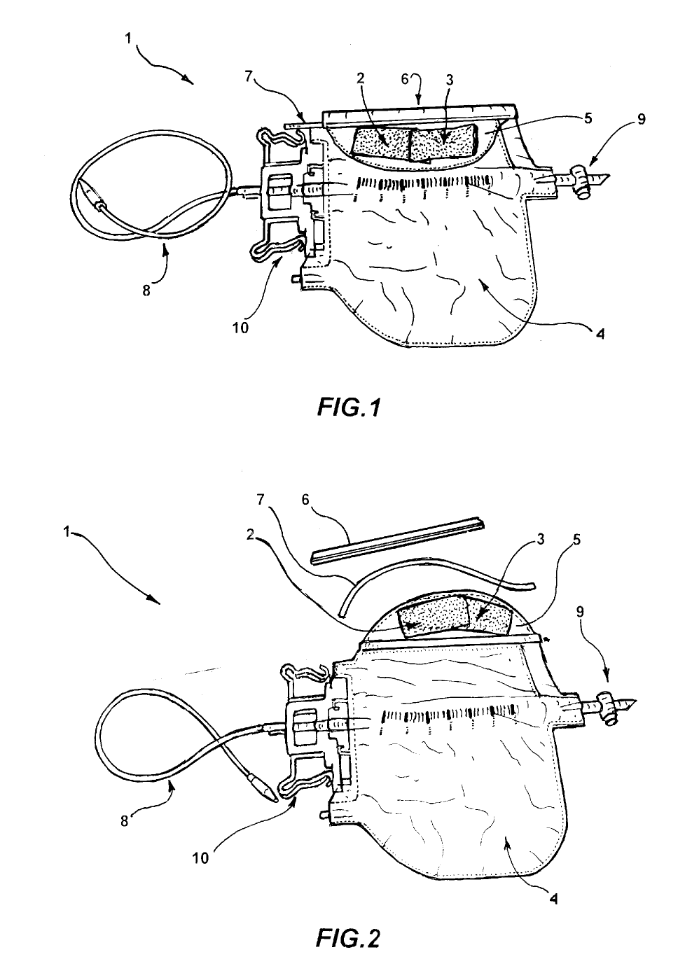

FIG. 1 is an overall view of the bag according to a preferred embodiment of the present invention.

FIG. 2 is an overall view of the same embodiment of FIG. 1 wherein some elements of the bag are shown separated and not directly connected to the bag itself.

The present invention will be described in details hereinafter by referring to the above-mentioned figures.

By firstly referring to FIG. 1, this represents an overall view showing, as a whole, a bag 1 according to the present invention, according to a preferred embodiment.

The bag of the present invention is a bag suitable to include liquid organic waste, such as for example urines, thoracic, abdominal drainages, ascites, plasma blood.

The bag includes inside thereof a first powder 2 capable of releasing the chlorine dioxide biocide into the liquid organic waste when the bag is filled up.

Advantageously two substances will be used which, once mixed in solution, release chlorine dioxide, the two substances are sodium chlorite and sodium bisulphate.

This powder bi-component is available under the trade name of TwinOxide®.

The bag 1 advantageously could comprise a second powder 3 apt to absorb the liquid organic waste. Examples of powders suitable to absorb the liquid included in the bag are super absorbent powders with absorption capacity comprised between 60 and 500 times the weight of liquids, such as for example the cross-linked sodium polyacrylate, preferably equipped with system aromatizer, for example the one available under the tradename of Sgaiel. Such powders are able to transform the organic liquid, in particular the urine into gel. Under equipped with “system aromatizer” it is meant that the substance absorbent itself can be perfumed or mixed to an additional perfumed substance.

Surprisingly it has been observed that the time for solidifying/gelatinizing the liquid included in the bag, in particular urine, is sufficient to make that chlorine dioxide determines the biocidal action thereof.

The above-described powders will be included in sacks, preferably water-soluble sacks which when come in contact with the liquid included in the bag dissolve, thus by letting that the powders come into solution. In an alternative embodiment means for breaking the sack could be provided, constituted by a bar. The bar could be made of any suitable material and shape so that the bar can be broken off into one or more portions by an operator. Once broken-off, the bar should produce a cutting surface able to break the sack, but not the outer bag 1.

The bag of the present invention, for example, could be a disposable bag made of polyethylene, biodegradable polyethylene or pvc and it could be both a primary and a secondary bag.

In the present description, under the term primary bag any bag is meant suitable to include liquid organic waste, in particular urine, connected or connectable to a catheter 8 and comprising means 9 for emptying the primary bag, for example standard connectors known to the person skilled in the art currently used for emptying this type of bags.

In the present description under the term secondary bag any bag is meant suitable to include liquid organic waste, in particular urine, comprising means apt to connect the secondary bag to a primary bag so that when the primary bag is filled up with organic liquid, the organic liquid included in the primary bag can be emptied into the secondary bag. Said means, for example, comprises a universal connector which can be connected to means for emptying the primary bags.

According to an embodiment the bag 1 further comprises means 6,7 apt to separate reversibly a first portion 4 from a second portion 5 of the bag. According to this embodiment the first portion 4 is filled up with liquid, such as urine or others, without coming in contact with the first powder 2 and/or second powder 3 which, on the contrary, is included in the second portion 5. Once the operator removes the separation of the two portions, the powders will enter in contact with the liquid by determining the disinfection and solidification/gelification thereof.

According to a preferred embodiment represented in FIGS. 1 and 2, the separation means comprises a first element 7, preferably flexible and with cylindrical shape, extending along the bag 1 by defining the two portions to be separated and a second element 6 comprising a portion which hooks to the first element 7 so as to separate reversibly the first portion 4 from the second one 5. As it is clear from FIG. 1 when the operator wishes to eliminate the separation between the two portions, he/she will take off the first element, by making that the second element 6 detaches and the two portions are no more separated.

A subject of the present invention is also a kit comprising one or more bags according to the present invention and at least a primary bag and/or a secondary bag.

The present invention has been sofar described with reference to some embodiments thereof. It is to be meant that other embodiments belonging to the same inventive core may exist, all within the protective scope of the herebelow reported claims.

1. A bag for liquid organic waste, containing inside thereof a first powder apt to release a biocide into said liquid organic waste, wherein said biocide is chlorine dioxide.

2. The bag according to claim 1, wherein said first powder comprises two substances which mixed in solution release chlorine dioxide.

3. The bag according to claim 2 wherein said two substances are sodium chlorite and sodium bisulphate.

4. The bag according to claim 1 further containing inside thereof a second powder apt to absorb said liquid organic waste.

5. The bag according to claim 4, wherein said second powder is a powder absorbent with absorption capacity comprised between 60 and 500 times a weight of the liquid organic waste to be absorbed.

6. The bag according to claim 4, wherein said second powder is an absorbent substance apt to transform urine into gel.

7. The bag according to claim 6 wherein said absorbent substance apt to transform urine into gel is a cross-linked sodium polyacrylate.

8. The bag according to claim 4, wherein said second powder further comprises a system aromatizer.

9. The bag according to claim 4, wherein said first and/or second powder are included into a sack.

10. The bag according to claim 4, further comprising means apt to separate reversibly a first portion of said bag from a second portion of said bag, wherein in said second portion said first powder and said second powder are included.

11. The bag according to claim 10, wherein said means comprises a first element extending along said bag and a second element comprising a portion apt to hook said first element so as to separate reversibly said first portion from said second portion.

12. The bag according to claim 1, wherein said bag is made of polyethylene, biodegradable polyethylene, or pvc.

13. The bag according to claim 1, wherein said liquid waste are urines, abdominal drainages, ascites, blood, or plasma.

14. The bag according to claim 1, further comprising a catheter connected or connectable to said bag.

15. The bag according to claim 9, wherein said bag further comprises means for breaking said sack.

16. The bag according to claim 9, wherein said sack is water-soluble.

17. The bag according to claim 1, wherein said bag is a primary bag or a secondary bag.

18. A kit comprising one or more bags according to claim 1 and at least a primary bag and/or a secondary bag.
